(19) United States
(12) Patent Application Publication
(10) Pub. No.: US 2021/0383408 A1
(43) Pub. Date: Dec. 9, 2021

(54) PROCESSING BENEFIT ELIGIBILITY DATA

(71) Applicant: IDENTITY THEFT GUARD SOLUTIONS, INC., Portland, OR (US)

(72) Inventor: Brent VanLoo, Forest Grove, OR (US)

(21) Appl. No.: 16/895,049

(22) Filed: Jun. 8, 2020

(51) Int. Cl. G06Q 30/00; G06Q 10/10; G06Q 50/26; G16H 40/20; G06Q 20/40; G06Q 30/04; G06Q 40/08; H04L 29/06; H04L 29/12; H04L 12/58

(52) U.S. Cl. CPC G06Q 30/0185; G06Q 10/10; G06Q 50/265; G16H 40/20; H04L 51/22; G06Q 30/04; G06Q 40/08; H04L 63/102; H04L 61/307; G06Q 20/40

(57) ABSTRACT

A provider of a service (e.g., a medical service) submits a request for authorization to bill for the service (e.g., a request for authorization to bill an insurance company of the recipient of the service). The recipient of the request (e.g., a medical services clearinghouse) informs a fraud prevention of the request. The fraud prevention communicates (e.g., by text message or email) with a computing device (e.g., a cell phone or desktop computer) associated with the recipient of the service to determine if the request is legitimate. If the request is not legitimate, the clearinghouse, the insurance company, the medical service provider or any suitable combination thereof is notified of the issue. By taking action on this notification, benefit eligibility fraud is avoided.

### TECHNICAL FIELD

The subject matter disclosed herein generally relates to computerized methods and systems for processing benefit eligibility data. Specifically, in some example embodiments, the present disclosure addresses systems and methods for coordinating multiple systems in processing benefit eligibility data.

### BRIEF DESCRIPTION OF THE DRAWINGS

Some embodiments are illustrated by way of example and not limitation in the figures of the accompanying drawings.

FIG. 1 is a network diagram illustrating a network environment suitable for processing benefit eligibility data, according to some example embodiments.

FIG. 2 is an architectural diagram illustrating components of a fraud prevention server in communication with other systems, according to some example embodiments.

FIG. 3 is a block diagram illustrating a user interface for verifying a user's identity, according to some example embodiments.

FIG. 4 is a block diagram illustrating a user interface for requesting a confirmation of an insurance verification request, according to some example embodiments.

FIG. 5 is a block diagram illustrating a database schema suitable for supporting processing benefit eligibility data, according to some example embodiments.

FIG. 6 is a flowchart illustrating operations of a computing device in performing a method of confirming a user's identity, according to some example embodiments.

FIG. 7 is a flowchart illustrating operations of a computing device in performing a method of detecting a fraudulent request for billing authorization, according to some example embodiments.

FIG. 8 is a swim-lane diagram illustrating communications between computer systems in performing a method of processing benefit eligibility data, according to some example embodiments.

FIG. 9 is a block diagram illustrating an example of a software architecture that may be installed on a machine, according to some example embodiments.

FIG. 10 is a diagrammatic representation of a machine in the form of a computer system within which a set of instructions may be executed for causing the machine to perform any one or more of the methodologies discussed herein, according to an example embodiment.

### DETAILED DESCRIPTION

Example methods and systems are directed to processing benefit eligibility data. Examples merely typify possible variations. Unless explicitly stated otherwise, components and functions are optional and may be combined or subdivided, and operations may vary in sequence or be combined or subdivided. In the following description, for purposes of explanation, numerous specific details are set forth to provide a thorough understanding of example embodiments. It will be evident to one skilled in the art, however, that the present subject matter may be practiced without these specific details.

A provider of a service (e.g., a medical service) submits a request for authorization to bill for the service (e.g., a request for authorization to bill an insurance company of the recipient of the service). The recipient of the request (e.g., a medical services clearinghouse) informs a fraud prevention of the request. The fraud prevention communicates with a computing device (e.g., a cell phone) associated with the recipient of the service to determine if the request is legitimate.

If the request is not legitimate, the clearinghouse, the insurance company, the medical service provider or any suitable combination thereof is notified of the issue. By taking action on this notification, benefit eligibility fraud is avoided.

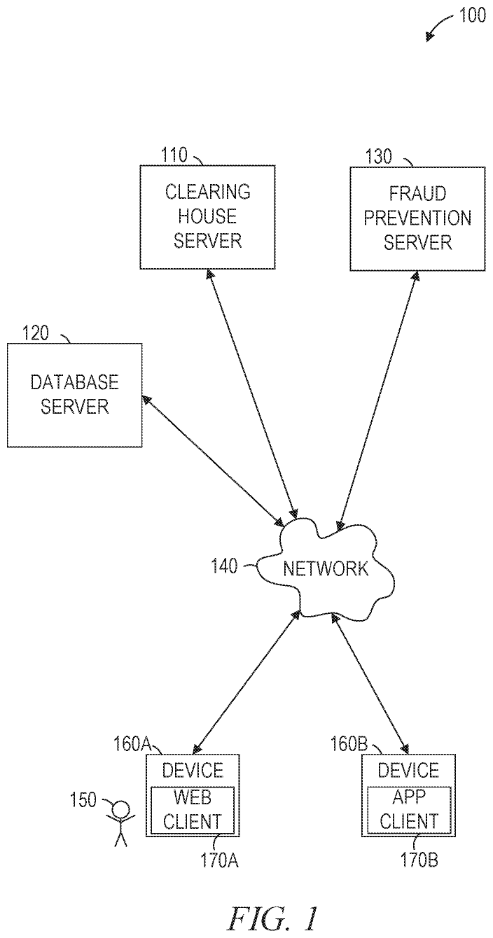

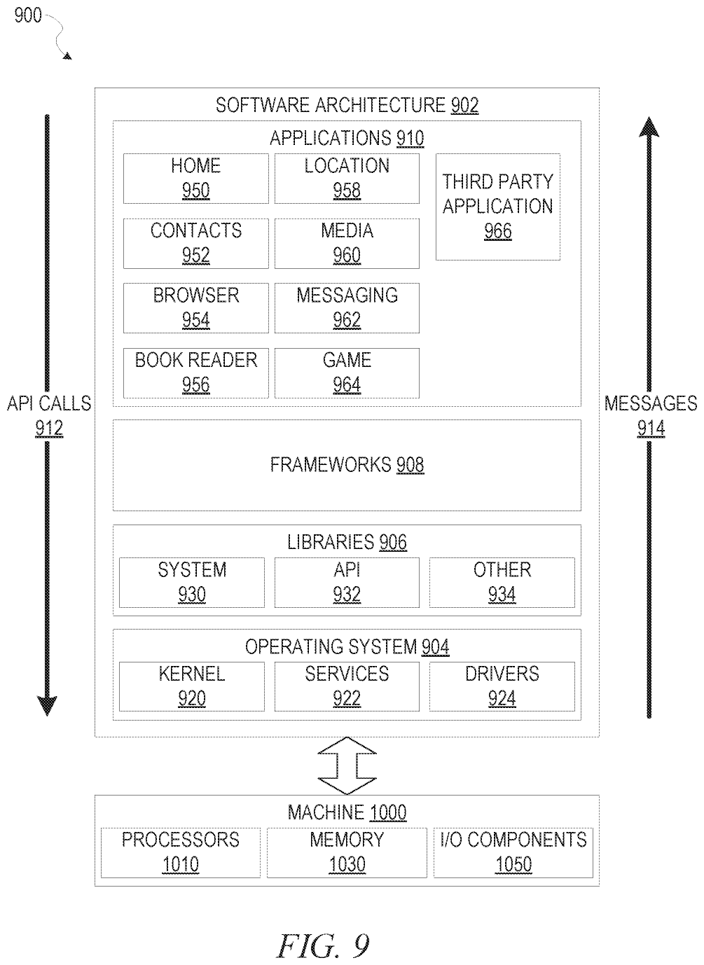

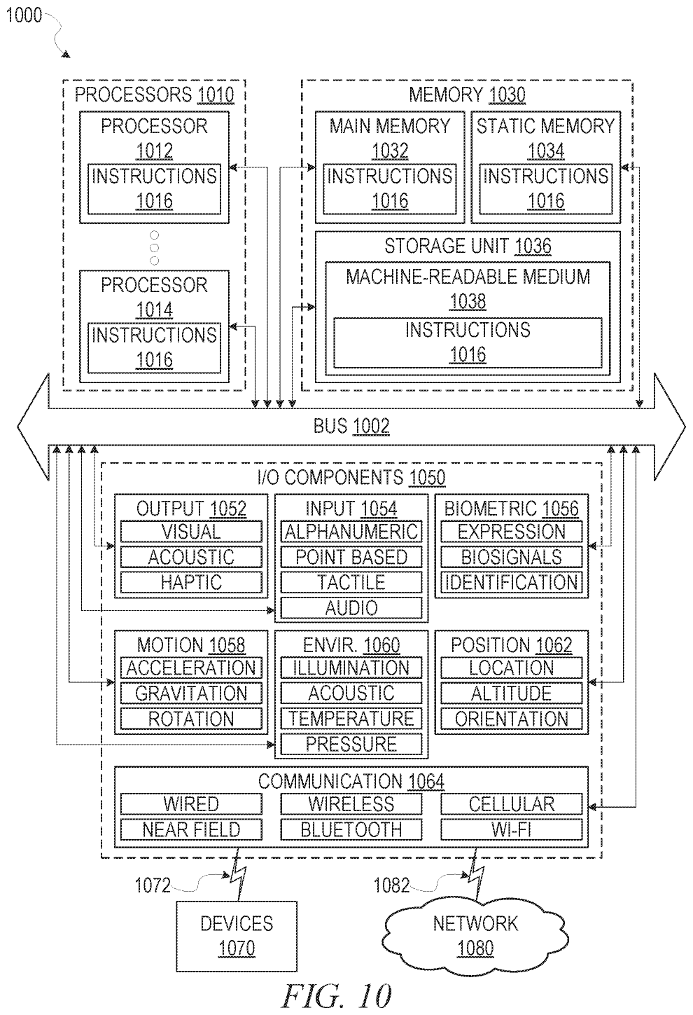

FIG. 1 is a network diagram illustrating a network environment 100 suitable for processing benefit eligibility data, according to some example embodiments. The network environment 100 includes a clearinghouse server 110, a database server 120, a fraud prevention server 130, and devices 160A and 160B, all communicatively coupled to each other via a network 140. The devices 160A and 160B may be collectively referred to as “devices 160,” or generically referred to as a “device 160.” The clearinghouse server 110, database server 120, and fraud prevention server 130 are network-based systems. The devices 160 may interact with the servers 110-130 using a web client 170A or an app client 170B. The servers 110-130 and the devices 160 may each be implemented in a computer system, in whole or in part, as described below with respect to FIGS. 9-10.

The clearinghouse server 110 collects data regarding transactions via the network 140. Many different insurance companies use unique protocols for requesting and providing authorization, receiving payment, and providing services. Thus, medical service providers face high overhead in interacting with multiple insurance companies. To alleviate this, a clearinghouse provides a single interface to users and handles the complexity of dealing with the multiple insurance companies. Intentionally or as a side effect of performing this intermediary function, the clearinghouse acquires information about the medical services being provided to medical consumers across multiple medical service providers and insurance companies.

Database services are provided by the database server 120 to one or more of the clearinghouse server 110 and the fraud prevention server 130. In some example embodiments, distinct database servers are used by each of the servers 110 and 130. The database services provided by the database server 120 include data storage and retrieval.

The fraud prevention server 130 provides one or more fraud preventions (e.g., health claims monitoring). Health claims monitoring provides an alert to a user whenever a medical claim is made against their identity, a request for billing authorization is made to their insurance company, or any suitable combination thereof.

Also shown in FIG. 1 is a user 150. The user 150 may be a human user (e.g., a human being), a machine user (e.g., a computer configured by a software program to interact with the devices 160 and one or more of the servers 110-130), or any suitable combination thereof (e.g., a human assisted by a machine or a machine supervised by a human). The user 150 is not part of the network environment 100, but is associated with the devices 160 and may be a user of the devices 160 (e.g., an owner of the devices 160A or 160B). For example, the device 160 may be a desktop computer, a vehicle computer, a tablet computer, a navigational device, a portable media device, or a smart phone belonging to the user 150. In some example embodiments, a user of a first device 160 is a medical services provider and a user of a second device 160 is a medical services recipient.

Any of the machines, databases, or devices shown in FIG. 1 may be implemented in a general-purpose computer modified (e.g., configured or programmed) by software to be a special-purpose computer to perform the functions described herein for that machine, database, or device. For example, a computer system able to implement any one or more of the methodologies described herein is discussed below with respect to FIGS. 9-10. As used herein, a “database” is a data storage resource and may store data structured as a text file, a table, a spreadsheet, a relational database (e.g., an object-relational database), a triple store, a hierarchical data store, or any suitable combination thereof. Moreover, any two or more of the machines, databases, or devices illustrated in FIG. 1 may be combined into a single machine, database, or device, and the functions described herein for any single machine, database, or device may be subdivided among multiple machines, databases, or devices.

The network 140 may be any network that enables communication between or among machines, databases, and devices (e.g., the fraud prevention server 130 and the devices 160). Accordingly, the network 140 may be a wired network, a wireless network (e.g., a mobile or cellular network), or any suitable combination thereof. The network 140 may include one or more portions that constitute a private network, a public network (e.g., the Internet), or any suitable combination thereof.

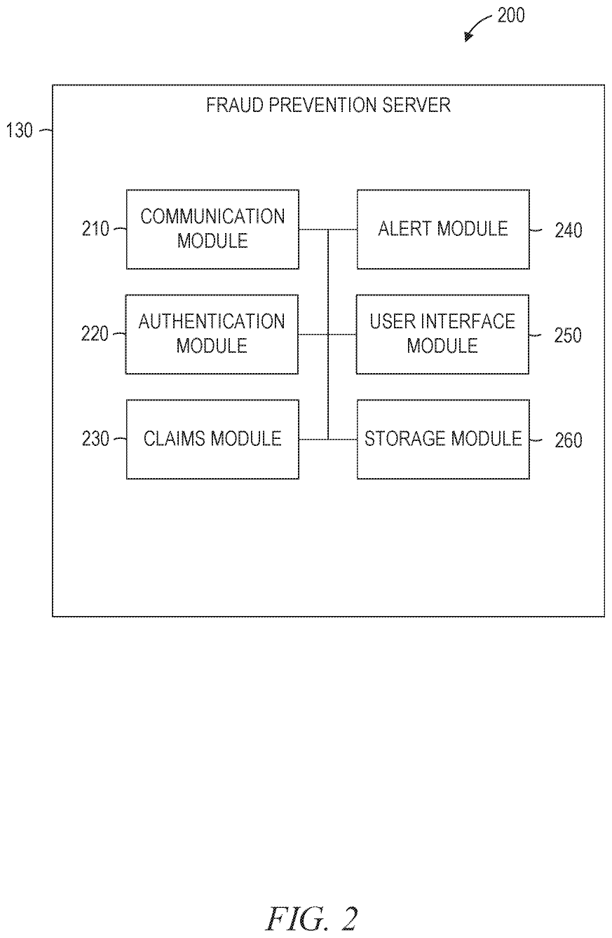

FIG. 2 is an architectural diagram 200 illustrating components of a fraud prevention server 130 in communication with other systems, according to some example embodiments. The fraud prevention server 130 includes a communication module 210, an authentication module 220, a claims module 230, an alert module 240, a user interface module 250, and a storage module 260, all configured to communicate with each other (e.g., via a bus, shared memory, a switch, or APIs). Any one or more of the modules described herein may be implemented using hardware (e.g., a processor of a machine) or a combination of hardware and software. For example, any module described herein may configure a processor to perform the operations described herein for that module. Moreover, any two or more of these modules may be combined into a single module, and the functions described herein for a single module may be subdivided among multiple modules. Furthermore, according to various example embodiments, modules described herein as being implemented within a single machine, database, or device may be distributed across multiple machines, databases, or devices.

The communication module 210 is configured to send and receive data. For example, the communication module 210 may receive from a medical service provider (e.g., using the device 160B), over the network 140, a request for authorization to bill a medical insurance company. The communication module 210 may provide the request to the user interface module 250, transmit a user interface provided by the user interface module 250 to a device of the user receiving the medical service (e.g., the device 160A), and receive user selections of options in the user interface for processing by the authentication module 220, the claims module 230, or the alert module 240; storage by the storage module 260; or any suitable combination thereof.

The authentication module 220 is configured to authenticate a user. For example, a question that only the user is expected to know the answer to may be presented to the user and the user authenticated only if the response is correct. In some example embodiments, multiple such questions are presented and the responses evaluated. The user may be permitted to proceed to view security status information, confirm or deny medical billing requests, or any suitable combination thereof, only after authentication is successful.

The claims module 230 is configured to determine if medical claims are valid and report, via the user interface module 250, the results of the determination. The alert module 240 is configured to generate alerts. The generated alerts may be provided to users (e.g., to report data access status changes, such as credit status changes, to report data breaches, to report suspected identity theft, or any suitable combination thereof), to medical service providers (e.g., to report rejection by a medical consumer of a request for authorization from the medical service provider), to insurance companies (e.g., to report attempted medical service fraud), or any suitable combination thereof. Each alert may be in the form of e-mail, text message, automated voice message, or another suitable method of notification.

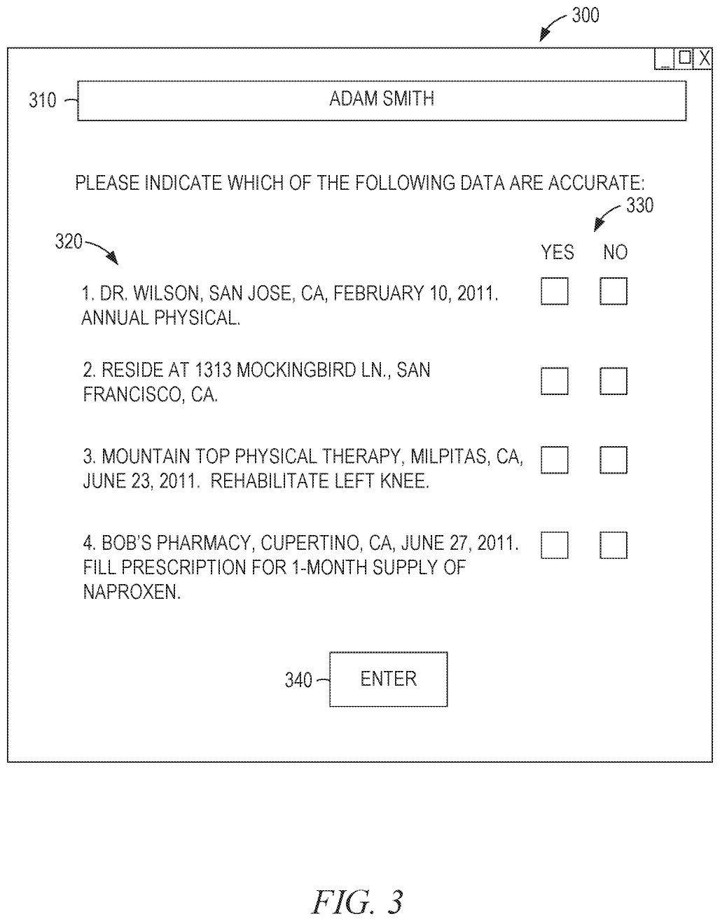

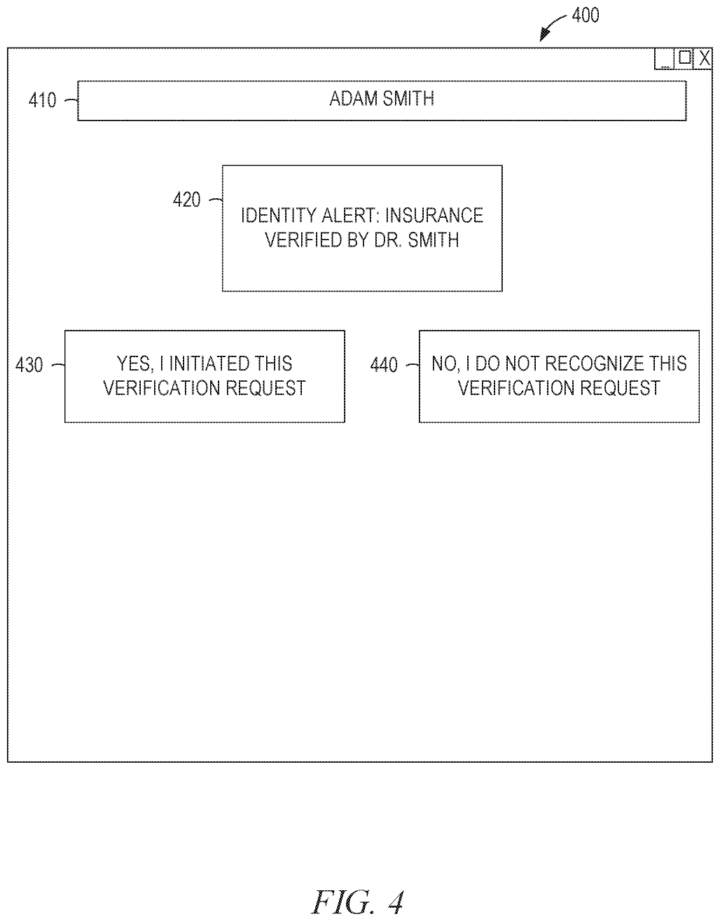

The user interface module 250 serves a website, via a hypertext transfer protocol (HTTP) connection, to the device 160A. The fraud prevention server 130 is in communication, via a representational state transfer (REST) application programming interface (API), with one or more of the servers 110 and 120. The user interface may include information regarding authenticating a user, determining if a request for medical service authorization is authentic, or any suitable combination thereof. For example, one or more of the user interfaces 300 and 400, described below with respect to FIGS. 3-4, may be presented by the user interface module 250, and selections may be received via an application interface or a web interface. The storage module 260 is configured to store data regarding users, entities, data access status, or any suitable combination thereof.

In some example embodiments, the database server 120 or the fraud prevention server 130 uses Structured Query Language (SQL) to access standard relational database and NoSQL to access databases other than standard relational databases. Dynamo NoSQL is a particular type of NoSQL based on key-value pairs. For example, a database may store usernames, passwords, authentication questions, user profiles, a user's name, social security number, birthdate, address, previous addresses, phone number, bank account numbers, or any suitable combination thereof.

FIG. 3 is a block diagram illustrating a user interface 300 for verifying a user's identity, according to some example embodiments. As can be seen in FIG. 3, the user interface 300 includes a name 310, prompts 320, check boxes 330, and button 340. The name 310 identifies the user being verified. Each of the prompts 320 is a fact or falsehood regarding the user. The checkboxes 330 allow the user to indicate whether each prompt is true or false.

The medical consumer may indicate which of the prompts are accurate and which are inaccurate by clicking on the appropriate check box 330. Once the accuracy of each prompt is indicated, the medical consumer may submit the responses by clicking the button 340. If the responses are correct, then the enrollment process may continue. In some example embodiments, the prompts 320 are aggregated from multiple sources. For example, in FIG. 3, the prompts numbered 1, 3, and 4 relate to medical transactions and the prompt 2 relates to an address of the user. Data for the medical transactions may be received by the fraud prevention server 130 from the clearinghouse server 110. Other data may be received from other servers (e.g., from a credit bureau server).

FIG. 4 is a block diagram illustrating a user interface 400 for requesting a confirmation of an insurance verification request, according to some example embodiments. As can be seen in FIG. 4, the user interface 400 includes the name 410, a status area 420, and the buttons 430 and 440. The name 410 identifies the user being verified. The status area 420 indicates the medical service provider that made the request on the identified user's identity. The buttons 430 and 440 allow the user to indicate if the request was initiated by the user or not. Thus, when the user goes to a doctor to request care and the doctor requests confirmation of the user's insurance, the user is notified of the request and can confirm that the request is valid. When another person goes to a doctor to request care and fraudulently presents the user's insurance information, the user is notified when the doctor requests insurance verification and can, by use of the button 440, inform the fraud prevention provider that the request is fraudulent. In this way, the medical services fraud can be detected immediately.

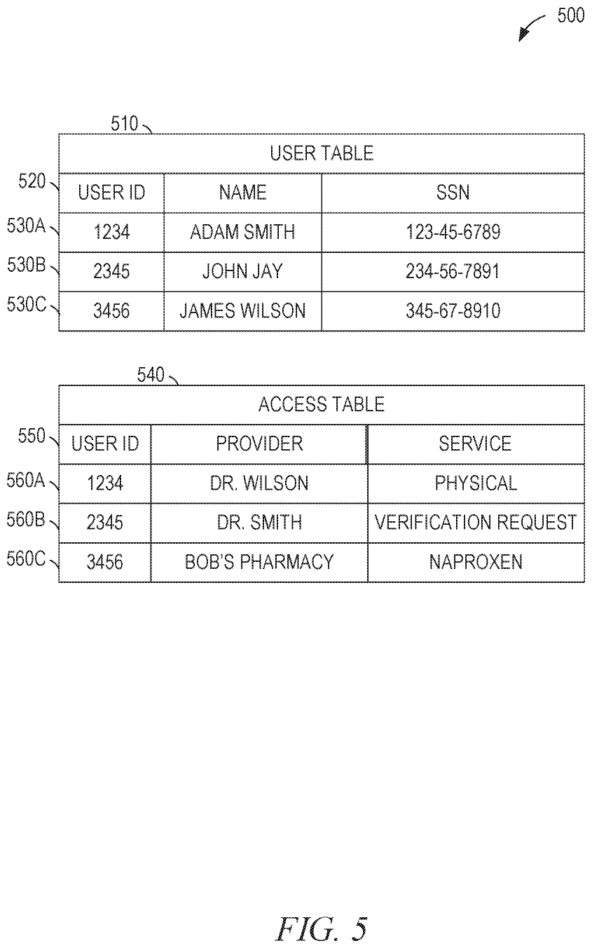

FIG. 5 is a block diagram illustrating a database schema 500 suitable for supporting processing benefit eligibility data, according to some example embodiments. The database schema 500 includes a user table 510 and an access table 540. The user table 510 is defined by a table definition 520, including a user identifier field, a name field, and a social security number (“SSN”) field, and includes rows 530A, 530B, and 530C. The access table 540 is defined by a table definition 550, including a user identifier field, a provider field, and a service field, and includes rows 560A, 560B, and 560C.

Each of the rows 530A-530C stores information for a user. The user identifier field stores a unique identifier for the user. The name field stores a name of the user. The SSN field stores a social security number for the user. In various example embodiments, additional or different fields are stored in the user table 510. For example, an address field, a birthdate field, a phone number field, or any suitable combination thereof may be stored.

Each of the rows 560A-560C stores data for an access of user data by a medical services provider. The row 560A shows that data (e.g., insurance validation) for the user with identifier 1234, “Adam Smith” of the user table 510, was requested by Dr. Wilson, who billed for a physical examination. According to the row 560B, the user with identifier 2345, “John Jay.” has had his insurance verified by Dr. Smith. The user with identifier 3456, “James Wilson,” as shown in the row 560C, has been billed for a Naproxen prescription by Bob's Pharmacy.

Data from the user table 510 may be used in performing fraud preventions. For example, user data such as name, username, password, phone number, street address, and the like may be stored in the user table 510 and used for requesting data from the clearinghouse server 110. Data from the access table 540 may be used to generate the user interface 400 to allow the user to confirm that access to data by medical providers is authorized.

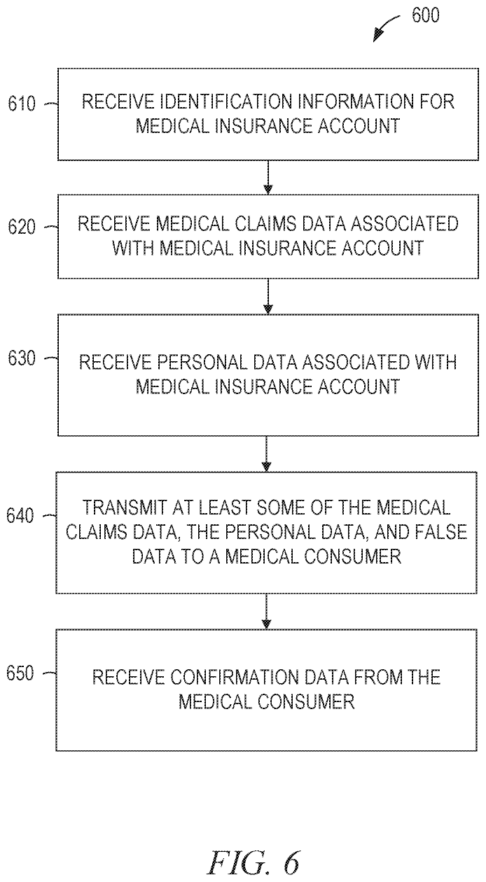

FIG. 6 is a flowchart illustrating operations of a computing device in performing a method 600 of confirming a user's identity, according to some example embodiments. The method 600 includes operations 610, 620, 630, 640, and 650. By way of example and not limitation, operations in the method 600 are described as being performed by the fraud prevention server 130, using modules described above with respect to FIG. 2.

In operation 610, the communication module 210 receives identification information for a medical insurance account. For example, the user 150 of the client device 160A may sign up for the medical fraud prevention by accessing a web page provided by the fraud prevention server 130 via the network 140, displayed on a screen of the device 160A by the web client 170A. The received identification information is stored in the user table 510.

The identification information may be provided by the medical consumer when, for example, the medical consumer signs up for a service offered by the fraud prevention service provider to reduce or prevent fraudulent use of the medical identity of the medical consumer. The medical consumer may provide the identification information online by accessing a website over the Internet, by sending an email, through a social media website, on paper by completing one or more forms and then transmitting the forms to a provider of the service, over the phone, or any other transmittal method known in the art now or in the future. In various embodiments, the identification information may be provided by a provider of medical insurance.

The identification information provided by the medical consumer may comprise any information that may be used to link the medical consumer with the medical insurance account of the medical consumer. In various embodiments, the medical identity may comprise any information shared between the medical consumer and the medical insurance provider that is providing medical insurance coverage to the medical consumer. The identification information may comprise general information about the medical consumer, such as name, home address, home telephone and cellular telephone number, electronic mail address, social media address, social security number, name and address of an employer of the medical consumer, or any suitable combination thereof.

The identification information may also comprise information specific to an insurance policy provided by the medical insurance provider to the medical consumer. The insurance policy information may comprise a name and address of the medical insurance provider, a policy number, a group number, a member number, a date that coverage under the insurance policy began, level of coverage, or any suitable combination thereof.

The communication module 210, in operation 620, receives medical claims data associated with the medical insurance account. For example, the clearinghouse server 110 provides, for validation purposes, at least a subset of the medical claims data stored by the clearinghouse server 110 to the fraud prevention server 130, via the network 140. In various embodiments, the medical claims data may be received from the medical insurance provider. In certain other embodiments, the medical claims data may be received from a provider of the medical goods or services.

The medical claims data may comprise any information or data that identifies particular medical goods or services reported against the medical insurance account of the medical consumer. For example, the medical claims data may comprise a name and address of the provider of the medical goods or services, such as the name of a doctor and the location of the office in which a medical service was provided. The medical claims data may also comprise a description of the goods or services provided. The description may comprise an alphanumeric medical billing code, such as Current Procedural Terminology (CPT) codes developed by the American Medical Association, or Healthcare Common Procedure Coding System (HCPCS) codes developed for Medicare use. The description may also comprise a diagnostic or procedural code, such as an International Statistical Classification of Diseases (ICD) codes. ICD codes may, for example, comprise a ICD-9-CM diagnostic code, a ICD-10-CM diagnostic code, or any other medical billing, diagnostic, or procedural code.

The description of the medical goods or services may also comprise a written description of the goods or service, such as a flu shot, a physical, a surgical procedure, a wheelchair, a prosthetic limb, a glucose test meter, a prescription medication, or any suitable combination thereof. The written description may comprise a description of the medical billing, diagnostic or procedural code. Additionally, the medical claims data may comprise the date or dates that the medical goods or services were provided.

While all medical goods or services are contemplated by the present disclosure, specific examples include: direct interaction with health practitioners, prescription medications, laboratory services, high technology diagnostic services, transportation by ambulance, blood supplies, eyeglasses, corrective lenses, external prosthetic devices, external orthotic devices, and internal medical devices.

Personal data associated with the medical insurance account is received by the communication module 210 (operation 630). For example, a credit monitoring bureau provides, for validation purposes, at least a subset of the personal data stored by a server of the credit monitoring bureau using the network 140.

In operation 640, the user interface module 250 transmits at least some of the medical claims data, the personal data, and false data to a medical consumer associated with the medical insurance account. For example, the user interface 300 may be presented on a display of the device 160A. The false data may be generated by the fraud prevention server 130, the clearinghouse server 110, the credit bureau server, or any suitable combination thereof.

The transmittal may occur via an email message, a message transmitted via a social media website, a telephone call, a letter, or any other transmittal method known in the art now or in the future. The transmittal may be in a secure mode to protect the privacy interests of the medical consumer. For example, the transmittal may be encoded such that a password may have to be entered prior to viewing. In various embodiments, the transmittal may be a notice that medical claims data have been received. In this situation, the medical consumer may securely log into a website (or make contact through another mechanism, such as a telephone call) and view the medical claims data.

The fraud prevention server 130, in operation 650, receives confirmation data from the medical consumer. For example, the medical consumer interacts with the user interface 300 to indicate which of the data items presented are true and which are false. Based on the user interaction correctly identifying the true and false elements, the user of the device 160A is confirmed to be the medical consumer.

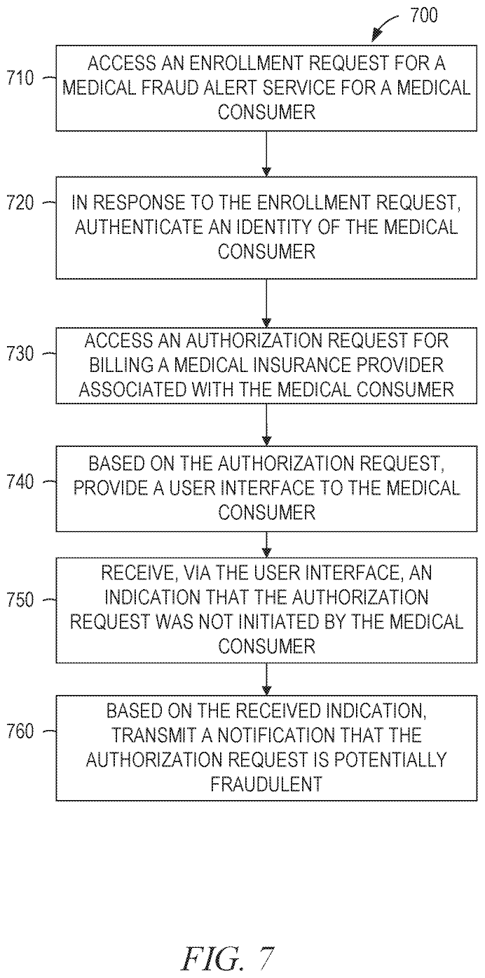

FIG. 7 is a flowchart illustrating operations of a computing device in performing a method 700 of detecting a fraudulent request for billing authorization, according to some example embodiments. The method 700 includes operations 710, 720, 730, 740, 750, and 760. By way of example and not limitation, operations in the method 700 are described as being performed by the fraud prevention server 130, using modules described above with respect to FIG. 2.

In operation 710, the fraud prevention server 130 accesses an enrollment request for a medical fraud alert service for a medical consumer. For example, the medical consumer may use a web browser to access a website served by the fraud prevention server 130. Using the website, the medical consumer provides identity information and requests the fraud prevention provider to provide the medical fraud alert service. Thus, in this example, the fraud prevention server accesses, via a network, the enrollment request from a first client device associated with the medical consumer. The enrollment request may include an identifier of a device (e.g., a phone number of a cellular phone) to which authentication requests are to be sent. Alternatively or additionally, the enrollment request may include other contact information, such as an email address, social media account name, or the like.

In response to the enrollment request, the authentication module 220 of the fraud prevention server 130 authenticates an identity of the medical consumer (operation 720). For example, the method 600 may be used to confirm that the user requesting the medical fraud alert service is actually the medical consumer being enrolled.

The claims module 230, in operation 730, accesses an authorization request for billing a medical insurance provider associated with the medical consumer. For example, a medical services provider may use the device 160B to submit an authorization request to bill a medical consumer's insurance provider. The authorization request is sent to a server of the insurance provider or to the clearinghouse server 110. The insurance provider or the clearinghouse forwards the authorization request, via the network 140, to the fraud prevention server 130. Alternatively, the authorization request is sent by the insurance provider or the clearinghouse to the database server 120 and the fraud prevention server 130 periodically accesses the stored authorization requests. In some example embodiments, the authorization request is an electronic data interchange eligibility and benefit inquiry (EDI 270).

In operation 740, based on the authorization request, the alert module 240 causes the user interface module 250 to provide a user interface to the medical consumer. For example, the user interface 400 may be presented, comprising information about the medical services provider, the service provided, the medical insurance provider, the amount of the billing request, or any suitable combination thereof. The user interface 400 may be presented on the device identified in the enrollment request. For example, using a phone number provided in the enrollment request, the user interface 400 may be presented as a text message or push notification to the medical consumer.

The transmittal may occur via an email message, a message transmitted via a social media website, a telephone call, a letter, or any other transmittal method known in the art now or in the future. The transmittal may be in a secure mode to protect the privacy interests of the medical consumer. For example, the transmittal may be encoded such that a password may have to be entered prior to viewing. In various embodiments, the transmittal may be a notice that medical claims data have been received. In this situation, the medical consumer may securely log into a website (or make contact through another mechanism, such as a telephone call) and view the medical claims data.

In operation 750, the fraud prevention server 130 receives, via the user interface, an indication that the authorization request was not initiated by the medical consumer. By way of example, the user may press or click on the button 440, causing an HTTP transmission of data from the user's device (e.g., the device 160A) to the fraud prevention server 130 via the network 140.

Based on the received indication, the alert module 240 transmits, to the clearinghouse server 110 or to an insurance provider server, a notification that the authorization request is potentially fraudulent (operation 760). Based on the notification, the clearinghouse server may notify the requesting medical service provider, deny the insurance authorization, request further instructions from the insurance provider, or any suitable combination thereof. Thus, in some example embodiments, the authorization request of operation 730 is sent from the medical services provider before a medical service is provided to the medical consumer or person attempting fraud in the medical consumer's name.

Alternatively, if the fraud prevention server 130 receives, via the user interface, an indication that the authorization request was initiated by the medical consumer, the insurance authorization request is allowed to proceed normally. Thus, the clearinghouse server 110 receives an authorization (or denial) from the insurance company server based on the information provided in the authorization request. In some example embodiments, the authorization response is an electronic data interchange eligibility and benefit inquiry response (EDI 271).

In some example embodiments, when the fraud prevention server 130 does not receive an indication from the medical consumer as to whether the authorization request was initiated by the medical consumer or not, a default action is taken after a predetermined period of time. The default action may be to allow the authorization request to proceed normally or to reject the authorization request.

The medical consumer may provide a variety of responses to the data. The medical consumer may confirm that the data are accurate. For example, the medical claims data may specify that a claim was made against the medical insurance account of the medical consumer for a visit to a certain doctor on a given date. If the medical consumer recognizes the visit as one that the medical consumer actually made, then the medical consumer may provide a confirmation that the medical claims data are accurate.

However, the medical consumer may not recognize the medical goods or services specified in the medical claims data. In this situation, the medical consumer may be reasonably certain that he or she did not receive the medical goods or services. For example, the medical consumer may be certain that he or she was out of town on the date specified and could not have received the medical goods or services on the date or at the location specified. Thus, the medical consumer may provide confirmation that the medical claims data are inaccurate.

The medical consumer also may not recognize the medical goods or services because the medical consumer cannot recall whether the medical claims data are accurate, particularly if a date specified in the medical claims data is not recent. For example, the medical claims data may indicate that the medical consumer visited a certain doctor six months ago. The medical consumer may recognize the doctor as being one that he or she has visited, but is unsure whether the date of the visit is correct. Here, the medical consumer may provide a notification that he or she is unable to determine the accuracy of the medical claims data.

In various embodiments, the confirmation status may comprise a notification that the medical consumer has viewed the data. For example, when the medical consumer opens an email message containing the data, a notification may be automatically sent that the email message was viewed. In other embodiments, the medical consumer may log onto a website to view the data. In this situation, a notification may be sent that the medical consumer accessed the website containing the data.

A variety of actions may then be taken based on the confirmation status received from the medical consumer. If the confirmation status indicates that the medical claims data are accurate, then no further action may be taken. In various embodiments, a notification may be sent to the medical insurance provider that the medical consumer confirmed the data as accurate. The medical insurance provider may then take appropriate action with the claim. In the situation where the medical consumer indicates that the data are inaccurate, then the data may be flagged as being potentially fraudulent. Further investigation into the claim may be warranted, and the medical insurance provider may be notified of this status.

When the medical consumer is unable to determine the accuracy of the data, this may or may not indicate a possible fraudulent claim. Therefore, the claim may be flagged as needing further investigation, and the medical insurance provider may be so notified. In various embodiments, further communications may be initiated with the medical consumer when the medical consumer either confirms that the data are inaccurate, or is unable to determine the accuracy of the medical claims data.

Another possible situation is that the medical consumer does not respond to the transmittal of the data. In various embodiments, the claim may be assumed to be accurate and the medical consumer neglected to respond as such. Alternatively, the medical claims data may be flagged for further investigation, and notice provided to the medical insurance provider. In various embodiments, a reminder may be transmitted to the medical consumer if no response is received after a predetermined period of time. The reminder may be transmitted by the fraud prevention service provider, or by another entity tasked with this responsibility.

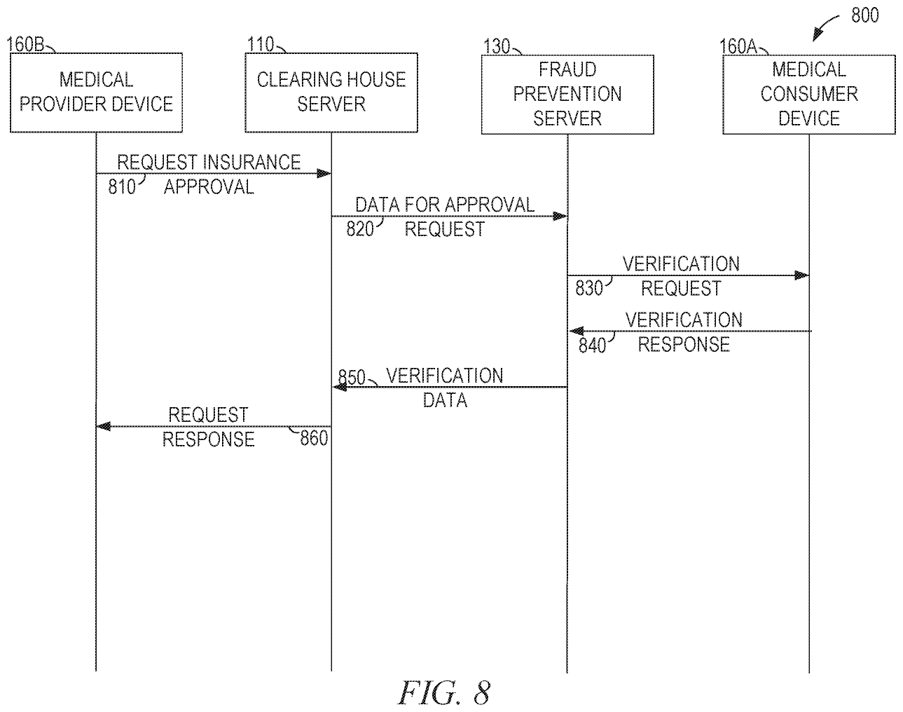

FIG. 8 is a swim-lane diagram 800 illustrating communications between computer systems in performing a method of processing benefit eligibility data, according to some example embodiments. The swim-lane diagram 800 shows communications 810, 820, 830, 840, 850, and 860 among the medical provider device 160B, the clearinghouse server 110, the fraud prevention server 130, and the medical consumer device 160A.

The medical provider device 160B submits a request for insurance approval to the clearinghouse server 110 in communication 810. For example, before seeing a patient, a doctor may submit a request to confirm that the insurance information provided by the patient is correct, and to request authorization from the insurance company to bill for the planned office visit. Rather than contacting the insurance company directly, the doctor may contact a clearinghouse, outsourcing the work of dealing with the various requirements of different insurance company's systems.

In communication 820, the clearinghouse server 110 sends data for the approval request to the fraud prevention server 130. For example, the name of the doctor and a description of the service to be performed may be sent.

The fraud prevention server 130 submits, in communication 830, a verification request to the medical consumer device 160A. As an example, the user interface 400 may be presented, including at least some of the data received in the communication 820.

In communication 840, the medical consumer device 160A provides a verification response to the fraud prevention server 130. For example, the user may press or click the button 430 or the button 440, causing the medical consumer device 160A to send an indication that the verification request was or was not initiated by the medical consumer.

The fraud prevention server 130 sends verification data, in communication 850, to the clearinghouse server 110. The verification data indicates whether the medical consumer verified the approval request or rejected it.

In communication 860, the clearinghouse server 110 sends a response to the insurance approval request. The response is based on the verification data. Thus, if the medical consumer denies initiating the request, the request is denied. In this way, medical insurance fraud attempted by using someone else's medical insurance information can be prevented before the medical service is provided. By preventing the fraud at an earlier stage than was possible using prior art methods, resources expended in handling the medical services transaction, detecting fraud, and rectifying the situation are saved.

When these effects are considered in aggregate, one or more of the methodologies described herein may obviate a need for certain efforts or resources that otherwise would be involved in detecting fraud. Computing resources used by one or more machines, databases, or devices (e.g., within the network environment 100) may similarly be reduced. Examples of such computing resources include processor cycles, network traffic, memory usage, data storage capacity, power consumption, and cooling capacity.

### Modules, Components, and Logic

Certain embodiments are described herein as including logic or a number of components, modules, or mechanisms. Modules may constitute either software modules (e.g., code embodied on a non-transitory machine-readable medium) or hardware-implemented modules. A hardware-implemented module is a tangible unit capable of performing certain operations and may be configured or arranged in a certain manner. In example embodiments, one or more computer systems (e.g., a standalone, client, or server computer system) or one or more processors may be configured by software (e.g., an application or application portion) as a hardware-implemented module that operates to perform certain operations as described herein.

In various embodiments, a hardware-implemented module may be implemented mechanically or electronically. For example, a hardware-implemented module may comprise dedicated circuitry or logic that is permanently configured (e.g., as a special-purpose processor, such as a field programmable gate array (FPGA) or an application-specific integrated circuit (ASIC)) to perform certain operations. A hardware-implemented module may also comprise programmable logic or circuitry (e.g., as encompassed within a general-purpose processor or other programmable processor) that is temporarily configured by software to perform certain operations. It will be appreciated that the decision to implement a hardware-implemented module mechanically, in dedicated and permanently configured circuitry, or in temporarily configured circuitry (e.g., configured by software) may be driven by cost and time considerations.

Accordingly, the term “hardware-implemented module” should be understood to encompass a tangible entity, be that an entity that is physically constructed, permanently configured (e.g., hardwired), or temporarily or transitorily configured (e.g., programmed) to operate in a certain manner and/or to perform certain operations described herein. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor may be configured as respective different hardware-implemented modules at different times. Software may accordingly configure a processor, for example, to constitute a particular hardware-implemented module at one instance of time and to constitute a different hardware-implemented module at a different instance of time.

Hardware-implemented modules can provide information to, and receive information from, other hardware-implemented modules. Accordingly, the described hardware-implemented modules may be regarded as being communicatively coupled. Where multiple of such hardware-implemented modules exist contemporaneously, communications may be achieved through signal transmission (e.g., over appropriate circuits and buses that connect the hardware-implemented modules). In embodiments in which multiple hardware-implemented modules are configured or instantiated at different times, communications between such hardware-implemented modules may be achieved, for example, through the storage and retrieval of information in memory structures to which the multiple hardware-implemented modules have access. For example, one hardware-implemented module may perform an operation, and store the output of that operation in a memory device to which it is communicatively coupled. A further hardware-implemented module may then, at a later time, access the memory device to retrieve and process the stored output. Hardware-implemented modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods described herein may be at least partially processor-implemented. For example, at least some of the operations of a method may be performed by one or more processors or processor-implemented modules. The performance of certain of the operations may be distributed among the one or more processors, not only residing within a single machine, but deployed across a number of machines. In some example embodiments, the processor or processors may be located in a single location (e.g., within a home environment, an office environment, or a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., application programming interfaces (APIs)).

### Electronic Apparatus and System

Example embodiments may be implemented in digital electronic circuitry, in computer hardware, firmware, or software, or in combinations of them. Example embodiments may be implemented using a computer program product, e.g., a computer program tangibly embodied in an information carrier, e.g., in a machine-readable medium for execution by, or to control the operation of, data processing apparatus, e.g., a programmable processor, a computer, or multiple computers.

A computer program can be written in any form of programming language, including compiled or interpreted languages, and it can be deployed in any form, including as a standalone program or as a module, subroutine, or other unit suitable for use in a computing environment. A computer program can be deployed to be executed on one computer or on multiple computers at one site or distributed across multiple sites and interconnected by a communication network.

In example embodiments, operations may be performed by one or more programmable processors executing a computer program to perform functions by operating on input data and generating output. Method operations can also be performed by, and apparatus of example embodiments may be implemented as, special-purpose logic circuitry, e.g., a field programmable gate array (FPGA) or an application-specific integrated circuit (ASIC).

The computing system can include clients and servers. A client and server are generally remote from each other and typically interact through a communication network. The relationship of client and server arises by virtue of computer programs running on the respective computers and having a client-server relationship to each other. In embodiments deploying a programmable computing system, it will be appreciated that both hardware and software architectures merit consideration. Specifically, it will be appreciated that the choice of whether to implement certain functionality in permanently configured hardware (e.g., an ASIC), in temporarily configured hardware (e.g., a combination of software and a programmable processor), or in a combination of permanently and temporarily configured hardware may be a design choice. Below are set out hardware (e.g., machine) and software architectures that may be deployed, in various example embodiments.

### Software Architecture

FIG. 9 is a block diagram 900 illustrating a software architecture 902, which may be installed on any one or more of the devices described above. FIG. 9 is merely a non-limiting example of a software architecture, and it will be appreciated that many other architectures may be implemented to facilitate the functionality described herein. The software architecture 902 may be implemented by hardware such as a machine 1000 of FIG. 10 that includes processors 1010, memory 1030, and I/O components 1050. In this example, the software architecture 902 may be conceptualized as a stack of layers where each layer may provide a particular functionality. For example, the software architecture 902 includes layers such as an operating system 904, libraries 906, frameworks 908, and applications 910. Operationally, the applications 910 invoke application programming interface (API) calls 912 through the software stack and receive messages 914 in response to the API calls 912, according to some implementations.

In various implementations, the operating system 904 manages hardware resources and provides common services. The operating system 904 includes, for example, a kernel 920, services 922, and drivers 924. The kernel 920 acts as an abstraction layer between the hardware and the other software layers in some implementations. For example, the kernel 920 provides memory management, processor management (e.g., scheduling), component management, networking, and security settings, among other functionality. The services 922 may provide other common services for the other software layers. The drivers 924 may be responsible for controlling or interfacing with the underlying hardware. For instance, the drivers 924 may include display drivers, camera drivers. Bluetooth® drivers, flash memory drivers, serial communication drivers (e.g., Universal Serial Bus (USB) drivers), Wi-Fi® drivers, audio drivers, power management drivers, and so forth.

In some implementations, the libraries 906 provide a low-level common infrastructure that may be utilized by the applications 910. The libraries 906 may include system libraries 930 (e.g., C standard library) that may provide functions such as memory allocation functions, string manipulation functions, mathematic functions, and the like. In addition, the libraries 906 may include API libraries 932 such as media libraries (e.g., libraries to support presentation and manipulation of various media formats such as Moving Picture Experts Group-4 (MPEG4). Advanced Video Coding (H.264 or AVC). Moving Picture Experts Group Layer-3 (MP3), Advanced Audio Coding (AAC). Adaptive Multi-Rate (AMR) audio codec. Joint Photographic Experts Group (JPEG or JPG), or Portable Network Graphics (PNG)), graphics libraries (e.g., an OpenGL framework used to render in two dimensions (2D) and three dimensions (3D) in a graphic context on a display), database libraries (e.g., SQLite to provide various relational database functions), web libraries (e.g., WebKit to provide web browsing functionality), and the like. The libraries 906 may also include a wide variety of other libraries 934 to provide many other APIs to the applications 910.

The frameworks 908 provide a high-level common infrastructure that may be utilized by the applications 910, according to some implementations. For example, the frameworks 908 provide various graphic user interface (GUI) functions, high-level resource management, high-level location services, and so forth. The frameworks 908 may provide a broad spectrum of other APIs that may be utilized by the applications 910, some of which may be specific to a particular operating system or platform.

In an example embodiment, the applications 910 include a home application 950, a contacts application 952, a browser application 954, a book reader application 956, a location application 958, a media application 960, a messaging application 962, a game application 964, and a broad assortment of other applications such as a third-party application 966. According to some embodiments, the applications 910 are programs that execute functions defined in the programs. Various programming languages may be employed to create one or more of the applications 910, structured in a variety of manners, such as object-orientated programming languages (e.g., Objective-C, Java, or C++) or procedural programming languages (e.g., C or assembly language). In a specific example, the third-party application 966 (e.g., an application developed using the Android™ or iOS™ software development kit (SDK) by an entity other than the vendor of the particular platform) may be mobile software running on a mobile operating system such as iOS™, Android™, Windows® Phone, or other mobile operating systems. In this example, the third-party application 966 may invoke the API calls 912 provided by the mobile operating system (e.g., the operating system 904) to facilitate functionality described herein.

### Example Machine Architecture and Machine-Readable Medium

FIG. 10 is a block diagram illustrating components of a machine 1000, according to some example embodiments, able to read instructions from a machine-readable medium (e.g., a machine-readable storage medium) and perform any one or more of the methodologies discussed herein. Specifically. FIG. 10 shows a diagrammatic representation of the machine 1000 in the example form of a computer system, within which instructions 1016 (e.g., software, a program, an application, an applet, an app, or other executable code) for causing the machine 1000 to perform any one or more of the methodologies discussed herein may be executed. In alternative embodiments, the machine 1000 operates as a standalone device or may be coupled (e.g., networked) to other machines. In a networked deployment, the machine 1000 may operate in the capacity of a server machine or a client machine in a server-client network environment, or as a peer machine in a peer-to-peer (or distributed) network environment. The machine 1000 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1016, sequentially or otherwise, that specify actions to be taken by the machine 1000. Further, while only a single machine 1000 is illustrated, the term “machine” shall also be taken to include a collection of machines 1000 that individually or jointly execute the instructions 1016 to perform any one or more of the methodologies discussed herein.

The machine 1000 may include processors 1010, memory 1030, and I/O components 1050, which may be configured to communicate with each other via a bus 1002. In an example embodiment, the processors 1010 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an Application-Specific Integrated Circuit (ASIC), a Radio-Frequency Integrated Circuit (RFIC), another processor, or any suitable combination thereof) may include, for example, a processor 1012 and a processor 1014 that may execute the instructions 1016. The term “processor” is intended to include multi-core processors that may comprise two or more independent processors (also referred to as “cores”) that may execute instructions contemporaneously. Although FIG. 10 shows multiple processors 1010, the machine 1000 may include a single processor with a single core, a single processor with multiple cores (e.g., a multi-core processor), multiple processors with a single core, multiple processors with multiple cores, or any combination thereof.

The memory 1030 may include a main memory 1032, a static memory 1034, and a storage unit 1036 accessible to the processors 1010 via the bus 1002. The storage unit 1036 may include a machine-readable medium 1038 on which are stored the instructions 1016 embodying any one or more of the methodologies or functions described herein. The instructions 1016 may also reside, completely or at least partially, within the main memory 1032, within the static memory 1034, within at least one of the processors 1010 (e.g., within the processor's cache memory), or any suitable combination thereof, during execution thereof by the machine 1000. Accordingly, in various implementations, the main memory 1032, the static memory 1034, and the processors 1010 are considered machine-readable media 1038.

As used herein, the term “memory” refers to a machine-readable medium 1038 able to store data temporarily or permanently and may be taken to include, but not be limited to, random-access memory (RAM), read-only memory (ROM), buffer memory, flash memory, and cache memory. While the machine-readable medium 1038 is shown in an example embodiment to be a single medium, the term “machine-readable medium” should be taken to include a single medium or multiple media (e.g., a centralized or distributed database, or associated caches and servers) able to store the instructions 1016. The term “machine-readable medium” shall also be taken to include any medium, or combination of multiple media, that is capable of storing instructions (e.g., instructions 1016) for execution by a machine (e.g., machine 1000), such that the instructions, when executed by one or more processors of the machine (e.g., processors 1010), cause the machine to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” shall accordingly be taken to include, but not be limited to, one or more data repositories in the form of a solid-state memory (e.g., flash memory), an optical medium, a magnetic medium, other non-volatile memory (e.g., Erasable Programmable Read-Only Memory (EPROM)), or any suitable combination thereof. The term “machine-readable medium” specifically excludes non-statutory signals per se.

The I/O components 1050 include a wide variety of components to receive input, provide output, produce output, transmit information, exchange information, capture measurements, and so on. In general, it will be appreciated that the I/O components 1050 may include many other components that are not shown in FIG. 10. The I/O components 1050 are grouped according to functionality merely for simplifying the following discussion and the grouping is in no way limiting. In various example embodiments, the I/O components 1050 include output components 1052 and input components 1054. The output components 1052 include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor), other signal generators, and so forth. The input components 1054 include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instruments), tactile input components (e.g., a physical button, a touch screen that provides location and force of touches or touch gestures, or other tactile input components), audio input components (e.g., a microphone), and the like.

In some further example embodiments, the I/O components 1050 include biometric components 1056, motion components 1058, environmental components 1060, or position components 1062, among a wide array of other components. For example, the biometric components 1056 include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 1058 include acceleration sensor components (e.g., accelerometer), gravitation sensor components, rotation sensor components (e.g., gyroscope), and so forth. The environmental components 1060 include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., machine olfaction detection sensors, gas detection sensors to detect concentrations of hazardous gases for safety or to measure pollutants in the atmosphere), or other components that may provide indications, measurements, or signals corresponding to a surrounding physical environment. The position components 1062 include location sensor components (e.g., a Global Positioning System (GPS) receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1050 may include communication components 1064 operable to couple the machine 1000 to a network 1080 or devices 1070 via a coupling 1082 and a coupling 1072, respectively. For example, the communication components 1064 include a network interface component or another suitable device to interface with the network 1080. In further examples, the communication components 1064 include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1070 may be another machine or any of a wide variety of peripheral devices (e.g., a peripheral device coupled via a USB).

Moreover, in some implementations, the communication components 1064 detect identifiers or include components operable to detect identifiers. For example, the communication components 1064 include Radio Frequency Identification (RFID) tag reader components. NFC smart tag detection components, optical reader components (e.g., an optical sensor to detect one-dimensional bar codes such as Universal Product Code (UPC) bar code, multi-dimensional bar codes such as Quick Response (QR) code. Aztec code. Data Matrix. Dataglyph, MaxiCode, PDF417, Ultra Code, Uniform Commercial Code Reduced Space Symbology (UCC RSS)-2D bar code, and other optical codes), acoustic detection components (e.g., microphones to identify tagged audio signals), or any suitable combination thereof. In addition, a variety of information can be derived via the communication components 1064, such as location via Internet Protocol (IP) geolocation, location via Wi-Fi® signal triangulation, location via detecting an NFC beacon signal that may indicate a particular location, and so forth.

### Transmission Medium

In various example embodiments, one or more portions of the network 1080 may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, the network 1080 or a portion of the network 1080 may include a wireless or cellular network and the coupling 1082 may be a Code Division Multiple Access (CDMA) connection, a Global System for Mobile communications (GSM) connection, or another type of cellular or wireless coupling. In this example, the coupling 1082 may implement any of a variety of types of data transfer technology, such as Single Carrier Radio Transmission Technology (1×RTT). Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS). High Speed Packet Access (HSPA), Worldwide Interoperability for Microwave Access (WiMAX), Long Term Evolution (LTE) standard, others defined by various standard-setting organizations, other long range protocols, or other data transfer technology.

In example embodiments, the instructions 1016 are transmitted or received over the network 1080 using a transmission medium via a network interface device (e.g., a network interface component included in the communication components 1064) and utilizing any one of a number of well-known transfer protocols (e.g., Hypertext Transfer Protocol (HTTP)). Similarly, in other example embodiments, the instructions 1016 are transmitted or received using a transmission medium via the coupling 1072 (e.g., a peer-to-peer coupling) to the devices 1070. The term “transmission medium” shall be taken to include any intangible medium that is capable of storing, encoding, or carrying the instructions 1016 for execution by the machine 1000, and includes digital or analog communications signals or other intangible media to facilitate communication of such software.

Furthermore, the machine-readable medium 1038 is non-transitory (in other words, not having any transitory signals) in that it does not embody a propagating signal. However, labeling the machine-readable medium 1038 as “non-transitory” should not be construed to mean that the medium is incapable of movement; the medium should be considered as being transportable from one physical location to another. Additionally, since the machine-readable medium 1038 is tangible, the medium may be considered to be a machine-readable device.

### Language

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the inventive subject matter has been described with reference to specific example embodiments, various modifications and changes may be made to these embodiments without departing from the broader scope of embodiments of the present disclosure. Such embodiments of the inventive subject matter may be referred to herein, individually or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

What is claimed is:

1. A method comprising:
accessing, at a fraud prevention server, from a first client device associated with a medical consumer, via a network, an enrollment request for a medical fraud alert service for the medical consumer;
in response to the enrollment request, authenticating, by the fraud prevention server, an identity of the medical consumer;
accessing, by the fraud prevention server via the network, an authorization request for billing, by a medical services provider, a medical insurance provider associated with the medical consumer;
based on the authorization request, providing, by the fraud prevention server, a user interface to the medical consumer via a second client device associated with the medical consumer;
receiving, at the fraud prevention server and via the user interface, an indication that the authorization request for medical billing was not initiated by the medical consumer; and
based on the received indication that the authorization request for medical billing was not initiated by the medical consumer, transmitting from the fraud prevention server to a server of the medical insurance provider, a notification that the authorization request is potentially fraudulent.

2. The method of claim 1, wherein:
the enrollment request comprises an email address; and
the providing of the user interface comprises sending an email to the email address.

3. The method of claim 1, wherein:
the second client device associated with the medical consumer is a mobile device; and
the providing of the user interface comprises generating a push notification on the mobile device.

4. The method of claim 1, wherein the enrollment request comprises an identifier of the second client device.

5. The method of claim 4, wherein:
the identifier of the second client device is a phone number; and
the user interface is presented via a text messaging service.

6. The method of claim 1, wherein:
the authorization request is transmitted from the medical services provider prior to providing medical care to the medical consumer.

7. The method of claim 1, wherein the accessing of the authorization request comprises:
receiving the authorization request from a server of a medical clearinghouse.

8. The method of claim 1, wherein the accessing of the authorization request comprises:
receiving the authorization request from a client device of the medical services provider.

9. The method of claim 1, further comprising:
determining that a predetermined period of time has elapsed after providing the user interface to the medical consumer via the second client device without receiving the indication that the authorization request for medical billing was not initiated by the medical consumer; and
based on the determination, sending a reminder to the second client device to respond to the provided user interface.

10. The method of claim 1, wherein the authorization request is an authorization request for billing for medical goods or services selected from the group consisting of direct interaction with health practitioners, prescription medications, laboratory services, high technology diagnostic services, transportation by ambulance, blood supplies, eyeglasses, corrective lenses, external prosthetic devices, external orthotic devices, and internal medical devices.

11. The method of claim 1, wherein the providing of the user interface to the medical consumer via the second client device associated with the medical consumer comprises receiving an access request from the second client device to access a website.

12. The method of claim 1, wherein the authorization request is an electronic data interchange eligibility and benefit inquiry (EDI 270).

13. A system comprising:
a memory that stores instructions; and
one or more processors configured by the instructions to perform operations comprising:
accessing, from a first client device associated with a medical consumer, via a network, an enrollment request for a medical fraud alert service for the medical consumer;
in response to the enrollment request, authenticating an identity of the medical consumer;
accessing, via the network, an authorization request for billing, by a medical services provider, a medical insurance provider associated with the medical consumer;
based on the authorization request, providing a user interface to the medical consumer via a second client device associated with the medical consumer;
receiving, via the user interface, an indication that the authorization request for medical billing was not initiated by the medical consumer; and
based on the received indication that the authorization request for medical billing was not initiated by the medical consumer, transmitting to a server of the medical insurance provider, a notification that the authorization request is potentially fraudulent.

14. The system of claim 13, wherein:
the enrollment request comprises an email address; and
the providing of the user interface comprises sending an email to the email address.

15. The system of claim 13, wherein:
the second client device associated with the medical consumer is a mobile device; and
the providing of the user interface comprises generating a push notification on the mobile device.

16. The system of claim 13, wherein the enrollment request comprises an identifier of the second client device.

17. The system of claim 16, wherein:
the identifier of the second client device is a phone number; and
the user interface is presented via a text messaging service.

18. A non-transitory machine-readable medium that stores instructions that, when executed by one or more processors, cause the one or more processors to perform operations comprising:
accessing, from a first client device associated with a medical consumer, via a network, an enrollment request for a medical fraud alert service for the medical consumer;
in response to the enrollment request, authenticating an identity of the medical consumer;
accessing, via the network, an authorization request for billing, by a medical services provider, a medical insurance provider associated with the medical consumer;
based on the authorization request, providing a user interface to the medical consumer via a second client device associated with the medical consumer;
receiving, via the user interface, an indication that the authorization request for medical billing was not initiated by the medical consumer; and
based on the received indication that the authorization request for medical billing was not initiated by the medical consumer, transmitting to a server of the medical insurance provider, a notification that the authorization request is potentially fraudulent.

19. The non-transitory machine-readable medium of claim 18, wherein:
the enrollment request comprises an email address; and
the providing of the user interface comprises sending an email to the email address.

20. The non-transitory machine-readable medium of claim 18, wherein:
the second client device associated with the medical consumer is a mobile device; and
the providing of the user interface comprises generating a push notification on the mobile device.
